(19) United States
(12) Patent Application Publication
(10) Pub. No.: US 2009/0000625 A1
(43) Pub. Date: Jan. 1, 2009

(54) Patient Arm Pad

(76) Inventor: David D. Alfery, Nashville, TN (US)

Correspondence Address: SWANSON & BRATSCHUN, L.L.C., 8210 SOUTHPARK TERRACE, LITTLETON, CO, 80120, US

(21) Appl. No.: 12/147,253

(22) Filed: Jun. 26, 2008

Related U.S. Application Data
(60) Provisional application No. 60/947,028, filed on Jun. 29, 2007.

(51) Int. Cl. A61F 5/37

(52) U.S. Cl. 128/878

(57) ABSTRACT

A patient arm support comprises a first elongate resilient segment extending along an axis having a top and a bottom surface, the bottom surface being configured to rest upon a substantially planar surface and the top surface being configured to support an upper portion of the patient's arm. A second elongate resilient segment extends along the axis and also has a top and a bottom surface, the bottom surface being configured to rest upon a substantially planar surface and the top surface being configured to support a lower portion of the patient's arm. In one embodiment a void provides axial separation between the top surfaces of the first and second resilient segments. The void is configured to receive the ulnar nerve at the elbow of the patient without the ulnar nerve contacting a surrounding surface. In another embodiment the second elongate resilient segment is inclined upward as it extends away from the first elongate resilient segment. In another embodiment a lengthwise channel is provided in the top surface of the second elongate resilient segment to receive and secure a patient's hand in a neutral position. Embodiments may include combinations of these features.

### RELATED APPLICATIONS

This application claims priority to U.S. application No. 60/947,028, filed on Jun. 29, 2007, entitled “Patient Arm Pad” which is incorporated herein by reference in its entirety.

### TECHNICAL FIELD

The present invention is directed to patient arm pads, and more particularly to patient arm pads for protecting one or more nerves in the vicinity of a patient's elbow or for providing enhanced patient comfort.

### BACKGROUND

Surgical patients undergoing operative procedures and medical patients being cared for in hospitals and clinics occasionally suffer from nerve injuries. Even when transient, they consume considerable financial and emotional resources, as patients undergo physical rehabilitation therapy in an attempt to regain function. When permanent, these injuries are devastating, leading to a tragic loss of patient functional abilities and autonomy.

Peripheral nerve injuries occur via two mechanisms: either a nerve is stretched beyond physiologic limits or it is compressed, with both causes leading to loss of ability to conduct nerve impulses. This in turn leads to loss of sensation and loss or motor function. In the most extreme injuries patients lose the ability to use the muscles of an extremity, lose sense of feeling, and may have excruciating sensations of pain emanating from the extremity.

Of peripheral nerves, the ulnar nerve is the nerve most often injured. This can occur either because the nerve is stretched (by sharply bending the arm at the elbow) or because pressure is applied at the area of greatest vulnerability, over the bony surfaces of the elbow (the area commonly referred to as “the funny bone” on the inside of the arm at the elbow). In an effort to prevent stretch injuries, practitioners make efforts to avoid prolonged periods of bending the arm greater than 90 degrees. A more difficult problem is trying to prevent pressure injuries to the ulnar nerve. When a patient is lying supine there is pressure applied over the bony surface of the back of the elbow simply because this is the area that the arm normally rests upon. This is precisely the area that is most vulnerable to compression injury for three reasons: 1) physiologic relative lack of natural fat and subcutaneous tissue to pad the elbow in this area, 2) the nerve traverses a relatively tight canal comprised of bone and fibrous tissue in this area, and 3) the vascular supply is often most marginal in this area. One theory is that compression injuries result from an inadequate blood supply to the nerve being compressed. In an effort to avoid compression injuries, surgical patients routinely have their elbows padded, with any of a variety of pads placed under the ulnar bony prominences at the elbow. Such padding is done in an effort to try to avoid any discrete area of pressure in this area of weight bearing. A variety of pads are used to provide this cushioning, including foam and gel pads, but they all have in common trying to distribute the area of contact in the vulnerable bony area to a soft padded surface that this area rests upon.

Other nerves in the upper arm are vulnerable to injury as well. The brachial nerve can be stretched beyond physiologic limits if the arm is allowed to hyperextend, and even in some very muscular patients simple normal extension of the arm to 180 degrees can cause stretch injury, as the brachial nerve may shorten over time if the arm is held in slight flexion for prolonged periods due to biceps muscle hypertrophy as occurs in some body builders. Finally, the radial nerve, running in a spiral manner on the outside of the upper arm, is vulnerable to pressure injury if localized pressure is applied over it for a prolonged period of time.

As indicated above, the most common way that attempts to prevent ulnar nerve injury is by padding the nerve at the elbows. Indeed, the American Society of Anesthesiologist's Practice Advisory for the Prevention of Perioperative Peripheral Neuropathies states that all anesthetized patients should have their elbows padded when undergoing surgery, leading to the almost universal adoption of this practice by anesthetists and operating room personnel. In addition, the Advisory calls for holding the hand in a neutral position when tucked at the patient's side, preventing hyperextension of the arm, and preventing pressure against the radial nerve.

No commercially available product to date addresses these recommendations. One known padding device is the Ulnar Nerve Protector distributed by Kendall Healthcare Products Company that is made of corrugated foam that is placed around a patient's arm in an attempt to provide padding at the elbow. See FIG. 1. Unfortunately, as illustrated in FIG. 2, the Kendall device results in pressure being specifically directed to and concentrated at the elbow. Thus, even with extensive use of the Kendall device and other available elbow pads, ulnar nerve injuries persist. Furthermore, the Kendall device fails to prevent hyperextension of the arm, does not hold the forearm in a neutral position and does not provide protection to the radial nerve.

The present invention is directed toward overcoming one or more of the problems discussed above.

### SUMMARY OF THE EMBODIMENTS

A first aspect is a patient arm support comprising a first elongate resilient segment extending along an axis having a top and a bottom surface, the bottom surface being configured to rest upon a substantially planar surface and the top surface being configured to support an upper portion of the patient's arm. A second elongate resilient segment extends along the axis and also has a top and a bottom surface, the bottom surface being configured to rest upon a substantially planar surface and the top surface being configured to support a lower portion of the patient's arm. A void provides axial separation between the top surfaces of the first and second resilient segments. The void is configured to receive the ulnar nerve at the elbow of the patient without the ulnar nerve contacting any surrounding surface. In one or more embodiments a third resilient segment underlies the void. In such an embodiment, the first, second and third resilient segments may each be integrally formed of a single resilient piece. In one or more embodiments the top surface of the second elongate resilient segment may be inclined upward as the top surface of the second elongate resilient segment extends axially from the void. In one or more embodiments the elongate resilient segments may be made of resilient foam. In such embodiments the resilient foam may be selected from the group consisting of open cell polyurethane, open cell ester, latex rubber, closed cell neoprene and closed cell polyethylene. The foam embodiments may include a top surface having an egg crate configuration. One or more embodiments may include at least one strap attached to one of the first and second elongate resilient segments, the strap being configured to attach a patient's arm to the elongate resilient segment. In addition, or alternatively, straps may be provided for attaching the patient arm support to a fixture. In one or more embodiments a lengthwise channel is formed in the upper surface of at least one of the first and second elongate resilient segments, the lengthwise channel being configured to help maintain a patient's arm on the upper surface.

Another aspect of the invention is a patient arm support comprising a first elongate segment extending along an axis having a top and bottom surface, the bottom surface being configured to rest upon a substantially planar surface and the top surface being configured to support an upper portion of the patient's arm. A second elongate resilient segment is attached to the first elongate resilient segment and extends along the axis. It also has a top and a bottom surface, the bottom surface being configured to rest upon a substantially planar surface and the top surface being configured to support a lower portion of a patient's arm. A lengthwise channel is in the top surface of the second elongate resilient segment, the lengthwise channel being configured to receive a portion of the patient's hand to secure the lower portion of the patient's hand against movement relative to the elongate resilient segment. The lengthwise channel may comprise a cut in the top surface of the second elongate resilient segment. In addition, a void may provide axial separation between the top surface of the first resilient segment and the top surface of the second resilient segment, the void being configured to receive the ulnar nerve at the elbow of the patient without contacting the surrounding surface. The top surface of the second elongate resilient segment may be inclined upward as the top surface extends axially from the first resilient segment. The elongate resilient segments may be made of a resilient foam.

A third aspect of the patient arm support comprises a first elongate resilient segment extending along an axis having a top and a bottom surface, the bottom surface being configured to rest upon the substantially planar surface and the top surface being configured to support an upper portion of the patient's arm. A second resilient segment is attached to the first resilient segment and extends along the axis and includes a top and a bottom surface. The bottom surface is configured to rest upon a substantially planar surface and the top surface is configured to support a lower portion of the patient's arm. The top surface of the second elongate resilient segment is inclined upward as the top surface extends axially away from the first elongate resilient segment. A lengthwise channel may be formed in the top surface of the second elongate resilient segment, the lengthwise channel being configured to receive a portion of the patient's hand therein. A lengthwise channel may further be formed in the top surface of the first elongate resilient segment.

Other aspects of the invention can include other combinations of the elements recited in the claims attached hereto.

### BRIEF DESCRIPTION OF THE DRAWINGS

FIG. 1 is a perspective view of an ulnar nerve protector distributed by Kendall Healthcare Products Company;

FIG. 2 is a photograph depicting the Kendall ulnar nerve protector in use with a patient;

FIG. 3 is a perspective view of a first embodiment of a patient arm pad;

FIG. 4 is a perspective view of a second embodiment of a patient arm pad;

FIG. 5 is a perspective view of a third embodiment of a patient arm pad;

FIG. 6 is a perspective view of a fourth embodiment of a patient arm pad;

FIG. 7 is a perspective view of a fifth embodiment of a patient arm pad; and

FIG. 8 is a side elevation view of a sixth embodiment of a patient arm pad.

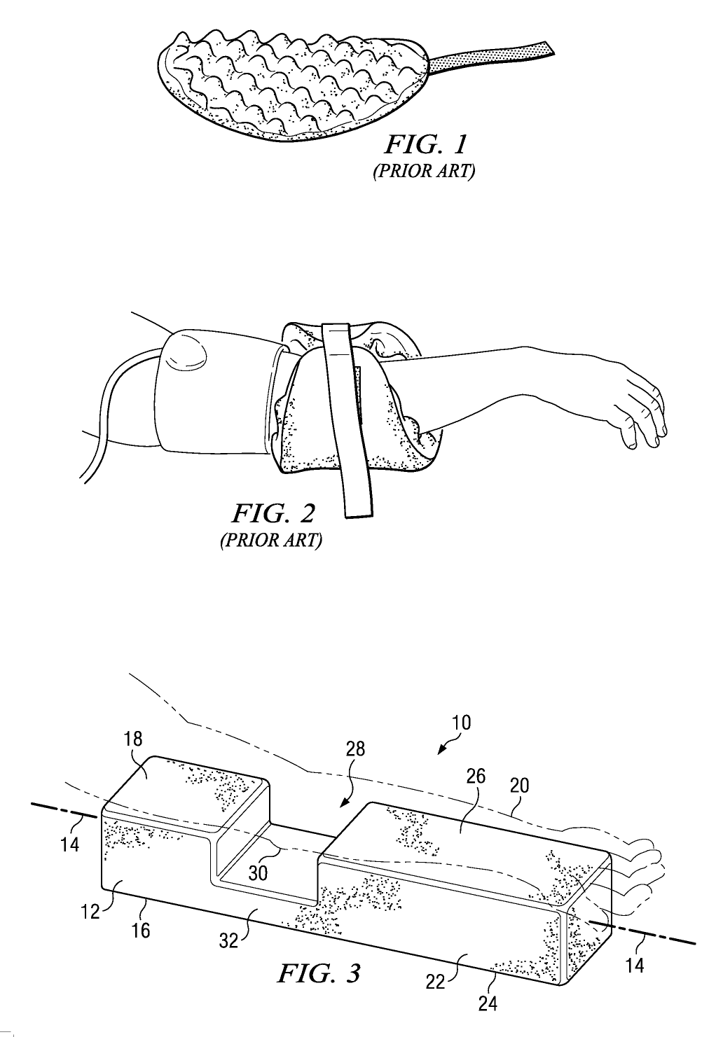

### DETAILED DESCRIPTION

FIG. 3 is a first embodiment of a patient arm support 10. In the first embodiment illustrated in FIG. 3, the patient arm support includes a first elongate resilient segment 12 which extends along an axis 14. The first elongate resilient segment 12 has a bottom surface 16 which is configured to rest upon a substantially planar surface. “Substantially planar” means a surface that is more or less flat, such as a hospital bed or surgical operating table or armboard. The first elongate resilient segment 12 further includes a top surface 18 which is configured to support an upper portion of a patient's arm 20 as illustrated in ghost lines in FIG. 3. The first embodiment further includes a second elongate resilient element 22 having a bottom surface 24 configured to rest upon a substantially planar surface and a top surface 26 configured to support a lower portion of a patient's arm 20. A void 28 axially separates the top surfaces 18, 26 of the first and second elongate resilient elements 12, 22. The void is configured to receive the ulnar nerve at the elbow 30 of the patient without contacting any surrounding surfaces, including the first and second elongate resilient elements. In other words, the elbow 30 is effectively supported in a sling of air. A third resilient element 32 may underlie the void 28 and connect the bottom surfaces 16, 24 of the first and second elongate resilient segments 12, 22. This third resilient segment 32 is intended to have a height such that the third resilient segment does not normally contact the elbow 30 of the patient and comes in contact with the elbow of the patient only under application of exceptional loads to the upper or lower arm of the patient.

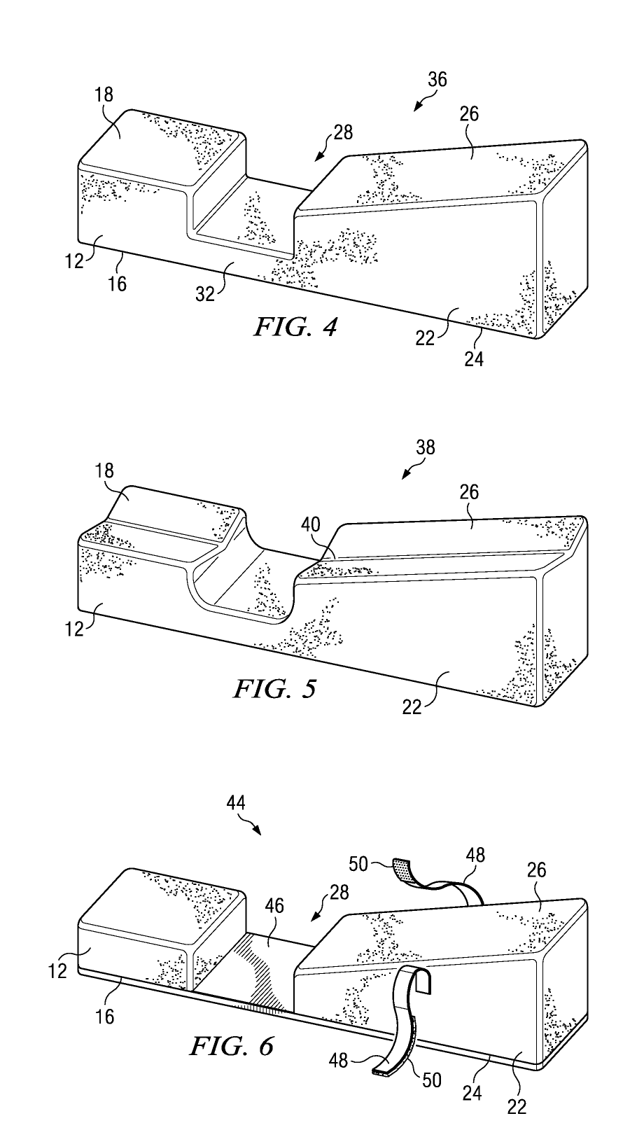

FIG. 4 illustrates a second embodiment of the pad support 36. Throughout this description the same reference numbers will be used to describe like elements of the various embodiments. In the embodiment illustrated in FIG. 4, the top surface 26 of the second elongate element 22 is inclined upward as the top surface extends axially away from the void 28. Although not illustrated, an alternative to the second embodiment would not include the void 28, or a removable plug could be provided in the void 28 to give the clinician the option of providing the void or not.

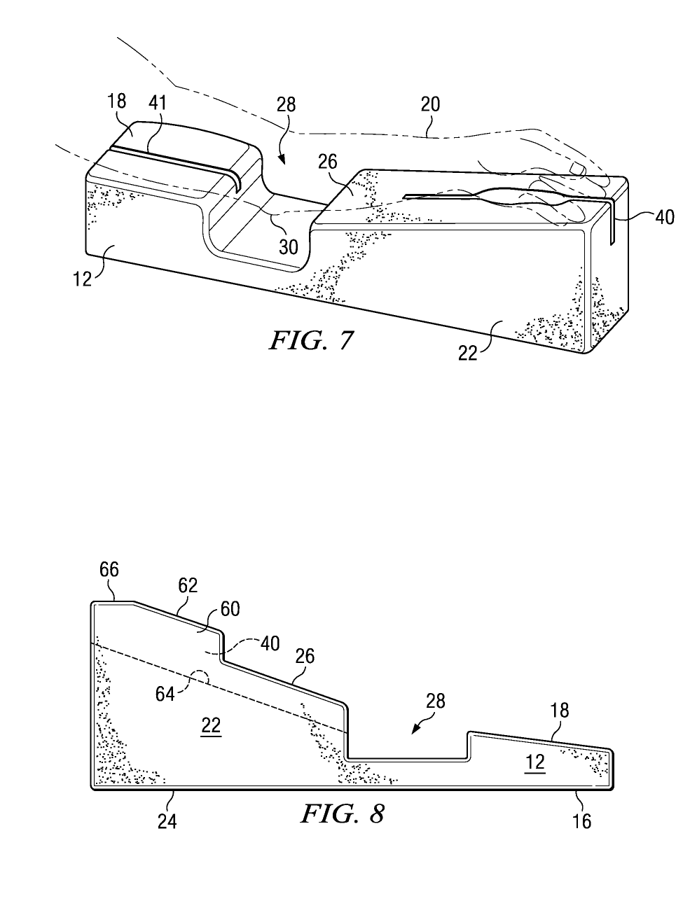

FIG. 5 is a third embodiment 38 of the patient arm support which is similar in configuration to the second embodiment 36, only the top surface 18 of the first elongate resilient segment 12 and the top surface 26 of the second elongate resilient segment 22 is formed with a lengthwise channel or groove 40 configured to help maintain a patient's arm on the top surfaces and the hand in a specific position. In the preferred embodiment the hand is held in a neutral position. The channel or groove depicted in FIG. 5 is a broad “v” shape. The channel could have other configurations functioning to center and secure a patient's arm on the pad and to minimize pressure directed against the radial nerve, such as along surface 18 of segment 12. In some embodiments the channel receives the pinky, perhaps the ring and middle fingers (or even all fingers) and the adjacent part of the hand to stabilize and secure the arm with the hand in a neutral position as illustrated in FIG. 7. For example, the lengthwise channel may be slit or cut in the foam formed by a knife or a narrow but deep channel (e.g., ⅛ inch wide and 1.5 inches deep). In the fifth embodiment illustrated in FIG. 7, such a cut or narrow but deep channel is shown at 40 in the top surface of the second elongate resilient segment 22. Optionally, and as illustrated in FIG. 7, a cut or narrow but deep channel 41 is provided in the top surface of the first elongate resilient segment 12. The channel could run along the entire length of each elongate resilient segment 12, 22, or only a portion of each segment. For example, the channel may be provided only toward a distal end of the second lengthwise segment 22, as illustrated in FIG. 7. A patient's arm 20 is shown in ghost lines in FIG. 7 with the pinky and ring finger and adjacent portion of the patient's hand received the lengthwise channel 40 in a neutral position. FIG. 7 shows deformation of the channel 40 and the bordering foam. Although not illustrated, an alternative to the third embodiment would not include the void 28, or a removable plug could be provided in the void 28 to give the clinician the option of providing the void or not.

FIG. 6 is a fourth embodiment 44 of a patient arm support. In this embodiment, the bottom surface 16 of the first elongate resilient element 12 and the bottom surface 24 of the second elongate element 22 are attached to a plate 46 with the void 28 overlying the plate 46. Also illustrated in the fourth embodiment 44 are straps 48 which may include hook and loop type fasteners 50 such as VELCRO® at the distal ends for securing a patients arm to a segment. Such straps could be provided on each segment of the arm support and multiple straps could be provided on each arm support of each of the embodiments described herein. Additional straps (not shown) could be provided for attaching the pad supports to a fixture as necessary or desired. Although not illustrated, an alternative to the forth embodiment would not include the void 28, or a removable plug could be provided in the void 28 to give the clinician the option of providing the void or not.

FIG. 8 is a sixth embodiment of the patient arm support featuring a fin 60 integrally formed toward a distal end of the second elongate resilient segment 22 and extending from the top surface 26. The fin 60 has a top surface 62 having a lengthwise channel 40, with the bottom of the channel 40 illustrated by ghost line 64. The channel 40 could, as illustrated, extend the entire length of the second elongate resilient segment 22 or only along the fin 60. The fin 60 is sized to receive the pinky, ring finger and perhaps more fingers and the adjacent portion of the patient's hand. By extending above the top surface 26 the channel 40 may receive the hand in a more natural, neutral and comfortable position than the fifth embodiment of FIG. 7, wherein the hand may need to bend at wrist slightly downward to be received in the channel 40. The sixth embodiment also illustrates that the angle of the top surface 18 of the first elongate resilient segment 12 and the top surface 26 of the second elongate resilient segment 22 relative to the bottom surfaces 16, 24 can vary as between the various embodiments as a matter of design choice. Thus, by way of example, the distal portion 66 of the second elongate resilient segment 22 is substantially flat, which may be desirable to support a patient's fingers. The top surfaces 62 and 26 have substantially the same incline, but these could be different as well. Finally, the top surface 18 of the first elongate resilient segment 12 is slightly inclined between its proximal end and its distal end (the end adjacent to the notch 28).

The embodiments illustrated in FIGS. 3-8 may be made of a resilient material such as a foam. Representative foams include open cell polyurethane, open cell ester, latex rubber, closed cell neoprene and closed cell polyethylene. The specific foam selected is a matter of design choice and is a function of the firmness and durability desired. Use of these foam materials would enable formation of the pad supports from a single integral piece of foam in a single manufacturing operation such as injection molding or stamping from a sheet of foam. Alternatively, the first and second elongate resilient segments 12, 22 may be made of other resilient materials such as gel based materials or other soft materials to provide desired support for the patient's arm. The term “resilient” is intended to mean a material which deforms at least temporarily under a load to conform to an arm disposed thereon or therebetween. Finally, the foam pad could be entirely replaced, if one desired, by an air-filled pad such as one made of a thin plastic inflatable material.

In the fourth embodiment 44 illustrated in FIG. 6, no padding is provided above the plate 46, but in other embodiments padding could be provided below the void 28. The plate 46 may be rigid, such as a metal plate or hard plastic or could be a flexible material such as rubber. The plate should be sufficiently robust to maintain the void 28 when the patient arm support is in service.

The second elongate resilient segment may be of a length to have the hand extend beyond its distal end as illustrated in FIG. 3 or alternatively could be long enough to provide support to the patient's hand. In the second and third embodiments illustrated in FIGS. 4 and 5, the angle of incline of the top surface 26 of the second elongate resilient element is approximately 14° from horizontal. This provides a slight flexion to the arm which may enhance patient comfort and help prevent stretch injuries to the patient's brachial nerve. Other angles of incline may be suitable to achieve these results as well. The dimensions of the various embodiments may vary depending upon patient size, physician preferences, and other factors. For example, in one embodiment the entire length (A) is 25 inches, the height at proximal end of the first segment 12 (B) is 4 inches, and height of distal end of the second segment (C) is 6 inches, the height at the void (D) is about 4 inches, the height of third segment 32 (E) is about 1.5 inches, the length of the first segment 12 (F) is 6 inches, the length of second segment 22 (G) is 14 inches, and the length of the third segment 32 (H) is 5 inches. In another embodiment, specifically the sixth embodiment of FIG. 8, the dimensions at A-H could be A=26 inches, B=2 inches, C=8 inches; D=4 inches at the distal end, 2.75 at the proximal end, E=1.5 inches, F=7 inches, G=14 inches and H=5 inches. The fin 60 may have a height of 1.5 inches and the length of the flat at the distal end of the second segment is 2 inches. A representative depth of the channel in any embodiment is in the range of 1.5-3 inches. These dimensions are only examples and are not intended to be limiting on the scope of the claims.

Another aspect of the present invention is a method of supporting the arm of a supine patient. The method may be practiced with one or more of the embodiments described herein. The method includes resting an upper portion of the arm of a patient on a first elongate resilient element. The lower portion of the arm of the patient is rested on a second elongate resilient element. A void is provided between the upper surface of the first and second resilient elements and the ulnar nerve at the elbow of the patient is positioned in the void so that the elbow does not contact surrounding surfaces and resides in the void. The method may further include inclining the upper surface of the second elongate resilient segment upward as it extends from the void to avoid stretching of the brachial nerve.

In those embodiments that are made of a resilient foam, the patient arm pads can be compressed to minimize their volume for shipping and storage. For example, the patient arm pads can be compressed into a compressed state and then contained in a package configured to maintain the resilient foam in its compressed state. One example of such packaging is a plastic envelope to which a vacuum is applied to remove essentially all the air. Thereafter, an opening of the envelope is heat sealed, resulting in the compressed patient arm pad being vacuum sealed in a compressed state within the envelope. Upon opening the envelope the availability of air to fill the voids of the foam allows the patient arm pad to expand to its operative form. Alternatively, the foam pad could be replaced entirely, if one wished, by an air-filled pad such as one made of a thin plastic inflatable material.

The various aspects and embodiments disclosed herein have a number of advantages over prior art patient arm pads. Embodiments including a void between the first and second segments for receiving the ulnar nerve at the elbow of the patient avoid pressure on the ulnar nerve by avoiding contact of the ulnar nerve with any surrounding surfaces, including the pad structure. Instead of allowing pressure on the ulnar never, the first and second elongate resilient segments support the upper and lower arm, which are anatomically better suited to distribute the weight of the arm. Those embodiments providing a third resilient segment underlying the void provide padding to the ulnar nerve in the event a patient's arm compresses the first or second elongate resilient segments causing the elbow to substantially fill the void. However, even under such circumstances, the pressure on the ulnar nerve is greatly reduced because the majority of the arm weight is supported by the first and second elongate resilient segments. In those embodiments wherein the second elongate resilient segment is inclined upward as it extends away from the void, the arm may be held in a more physiologically comfortable position than when fully extended. In addition to facilitating patient comfort, the inclined second segment may prevent excessive stretching and injury to the brachial nerve. Alternative embodiments not providing the inclined second segment may have advantages where a surgeon would find a flexed arm position to interfere with surgical exposure. Those embodiments of the invention with a longitudinal groove or notch on the upper surfaces of the elongate resilient segments help maintain the arm on the upper surface as well as hold the hand in a neutral position. Those embodiments including straps for securing the arm can further assure the patient's arm will remain on the top surface of the resilient segments. Those embodiments made of foam provide a patient arm support that can be manufactured from a single integral foam piece easily and inexpensively. A wide variety of foams can be used in placing the various embodiments to provide a desired support and patient comfort. Texturing such as an egg crate configuration can be added to the top surface of the foam as desired. Alternatively, the foam structure could be replaced, if one wished, by an air-filled pad such as one made of a thin plastic inflatable material.

Embodiments may have a removable plug filling the void 28. The removable plug may simply comprise material cut in the formation of the void 28. This would give the clinician the option of having the void or not. In addition, as described above, the various embodiments show with a void 28 could be made without the void. For example, if only the inclined second elongate portion was deemed important for a particular application or only the channel to secure and maintain the hand in a neutral position was deemed important for a particular application. Even in these configurations pressure would be relieved from the ulnar nerve at the elbow because the weight of the arm is distributed over such as large length of foam.

Various embodiments of the patient arm pad could also include permutations of the various elements recited in the claims as if each dependent claim was multiple dependent claim incorporating the limitations of each of the preceding dependent claims as well as the independent claims. Such permutations are expressly within the scope of this disclosure.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

What is claimed is:

1. A patient arm support comprising:
a first elongate resilient segment extending along an axis having at top and a bottom surface, the bottom surface being configured to rest upon a substantially planar surface and the top surface being configured to support an upper portion of the patient's arm;
a second elongate resilient segment extending along the axis having a top and a bottom surface, the bottom surface being configured to rest upon the substantially planar surface and the top surface being configured to support a lower portion of the patient's arm; and
a void providing axial separation between the top surface of the first resilient segment and the top surface of the second resilient segment, the void being configured to receive the ulnar nerve at the elbow of the patient without contacting a surrounding surface.

2. The patient arm support of claim 1 further comprising the first resilient segment and the second resilient segment being integrally formed from a single resilient piece.

3. The patient arm support of claim 1 further comprising a third resilient segment underlying the void.

4. The patient arm support of claim 3 further comprising the first, second and third resilient segments each being integrally formed from a single resilient piece.

5. The patient arm support of claim 1 further comprising the top surface of the second elongate resilient segment being inclined upward as the top surface extends axially from the void.

6. The patient arm support of claim 1 further comprising the elongate resilient segments being made of resilient foam.

7. The patient arm support of claim 6 wherein the resilient foam is selected from the group consisting of open cell polyurethane, open cell ester, latex rubber, closed cell neoprene and closed cell polyethylene.

8. The patient arm support of claim 6 wherein the top surface of the resilient foam has an egg crate configuration.

9. The patient arm support of claim 1 further comprising at least one strap attached to one of the first and second elongate resilient segments, the strap being configured to attach the patient's arm thereto.

10. The patient arm support of claim 1 further comprising a lengthwise channel formed in the upper surface of at least one of the first and second elongate resilient segments.

11. The patient arm support of claim 10 wherein the lengthwise channel comprises a cut in the top surface of the second elongate resilient segment.

12. The patient arm support of claim 5 further comprising a lengthwise channel in the top surface of the second elongate resilient segment.

13. The patient arm support of claim 12 further comprising a lengthwise channel formed in the top surface of the first elongate resilient segment.

14. The patient arm support of claim 1 further comprising a removable plug in the void configured to fill the void and transition between the top surfaces of the first and second elongate resilient segments.

15. The patient arm support of claim 6, further comprising the resilient foam being compressed in a compressed state to minimize its volume and contained with packaging configured to maintain the resilient foam in its compressed state.

16. The patient arm support of claim 15 wherein the packaging comprises a vacuum evacuated and heat-sealed sheets of plastic.

17. The patient arm support of claim 1 further comprising a fin extending from the top surface of the second elongate resilient segment near its distal end.

18. The patient arm support of claim 17 further comprising a lengthwise channel in a top surface of the fin configured to receive a portion of a patient's hand.

19. The patient arm support of claim 1 further comprising the elongate resilient segments being made of a thin plastic inflatable material.

20. A patient arm support comprising:
a first elongate resilient segment extending along an axis having at top and a bottom surface, the bottom surface being configured to rest upon a substantially planar surface and the top surface being configured to support an upper portion of the patient's arm;
a second elongate resilient segment attached to the first elongate resilient segment extending along the axis having a top and a bottom surface, the bottom surface being configured to rest upon the substantially plainer surface and the top surface being configured to support a lower portion of the patient's arm; and
a lengthwise channel in the top surface of the second elongate resilient segment, the lengthwise channel being configured to receive a portion of a patient's hand to secure the portion of the patient's hand against movement relative to the second elongate resilient segment.

21. The patient arm support of claim 20 wherein the lengthwise channel comprises a cut in the top surface of the second elongate resilient segment.

22. The patient arm support of claim 20 further comprising a void providing axial separation between the top surface of the first resilient segment and the top surface of the second resilient segment, the void being configured to receive the ulnar nerve at the elbow of the patient without contacting a surrounding surface.

23. The patient arm support of claim 20 further comprising the elongate resilient segments being made of resilient foam.

24. The patient arm support of claim 23 wherein the resilient foam is selected from the group consisting of open cell polyurethane, open cell ester, latex rubber, closed cell neoprene and closed cell polyethylene.

25. The patient arm support of claim 22 further comprising a removable plug in the void configured to fill the void and transition between the top surfaces of the first and second elongate resilient segments.

26. The patient arm support of claim 20 further comprising a lengthwise channel in the top surface of the first elongate resilient segment.

27. The patient arm support of claim 19 further comprising the elongate resilient segments being made of a thin plastic inflatable material.

28. A patient arm support comprising:
a first elongate resilient segment extending along an axis having at top and a bottom surface, the bottom surface being configured to rest upon a substantially planar surface and the top surface being configured to support an upper portion of the patient's arm; and
a second elongate resilient segment attached to the first elongate resilient segment extending along the axis having a top and a bottom surface, the bottom surface being configured to rest upon the substantially planar surface and the top surface being configured to support a lower portion of the patient's arm, the top surface of the second elongate resilient segment being inclined upward as the top surface extends axially away from the first elongate resilient segment.

29. The patient arm support of claim 28 further comprising a lengthwise channel formed in the top surface of the second elongate resilient segment, the lengthwise channel being configured to receive a portion of a patient's hand to secure the portion of the patient's hand against movement relative to the second elongate resilient segment.

30. The patient arm support of claim 29 further comprising a lengthwise channel formed in the top surface of the first elongate resilient segment.
